(19) United States
(12) Patent Application Publication
(10) Pub. No.: US 2011/0257473 A1
(43) Pub. Date: Oct. 20, 2011

(54) ADULT TOY ENHANCING FEMALE CLITORAL STIMULATION

(76) Inventor: Rosemarie Torla, Bellmore, NY (US)

(21) Appl. No.: 12/763,039

(22) Filed: Apr. 19, 2010

(51) Int. Cl. A61F 5/00

(52) U.S. Cl. 600/38

(57) ABSTRACT

An adult toy device having a clip-on portion and a wing portion. The clip-on portion includes a ring positionable so as to surround and extrude a female clitoris. The wing portion is attached to the clip-on portion and includes a pair of lips positionable adjacent to labia walls, a gap formed between the lips, and a pair of wings extending from the lips and having a resiliency. Further, the toy device includes a pair of finger gripping regions, each finger gripping region being positioned at one of the wings.

### BACKGROUND OF THE INVENTION

The invention generally relates to a device for increasing female sexual stimulation.

Clitoral vascular engorgement plays an important role in female sexual arousal and overall sexual satisfaction. Sexual arousal results in smooth muscle relaxation and arterial vasodilation within the clitoris. The resultant increase in blood flow leads to tumescence of the glans clitoris and increased sexual arousal.

A variety of devices have been developed for stimulating female sexual response. The most common devices are vibrators and other mechanical devices which create friction against the clitoris. For example, U.S. Pat. No. 6,179,775 to Thompson discloses a clitoral stimulation device in which a clitoral pad is provided with a u-shaped notch partially surrounding and touching the ventral aspect of the clitoris. The pad is designed to engage and stimulate the clitoris during the intercourse.

Further, U.S. Pat. No. 6,733,438 to Dann et al. discloses a clitoral stimulation device formed from a resilient, partially deformable material. The device is oval-shaped and is positionable into the anterior vaginal vestibul over the clitoris to be held in place by labial folds.

Finally, International Patent Application No. WO96/11660 to Fuchs discloses an orgasm aiding device having a flexible open ring shaped as an oval so as to rest against the clitoris.

### SUMMARY OF THE INVENTION

It is an object of the present invention to provide a device enhancing female sexual experience by extruding the clitoris and separating the labia allowing for maximum contact with the clitoris during regular sexual activity.

In one general aspect, the invention is an adult toy device having a clip-on portion and a wing portion. The clip-on portion includes a ring positionable so as to surround and extrude a female clitoris. The wing portion is attached to the clip-on portion and includes a pair of lips positionable adjacent to labia walls, a gap formed between the lips, and a pair of wings extending from the lips and having a resiliency. Further, the toy device includes a pair of finger gripping regions, each finger gripping region being positioned at one of the wings.

In another general aspect, the invention is a method of enhancing female orgasm. The method includes providing an adult toy device which includes a clip-on portion having a ring, a wing portion having a pair of lips, a gap formed between the lips, a pair of wings extending from the lips, and a pair of finger gripping regions. The method is accomplished by first, squeezing the wings of the adult toy device by pinching the finger gripping regions toward each other; then, positioning the ring of the clip-on portion around female clitoris so as to surround and extrude it; subsequently, positioning the lips of the wing portion adjacent to labia walls; and then, releasing the finger gripping regions so as to push the lips against the labia walls, expanding the gap between the lips and exposing a vaginal opening within the gap.

Other features and advantages of the present invention will become apparent from the following description of the invention which refers to the accompanying drawings.

### BRIEF DESCRIPTION OF THE DRAWINGS

The invention is illustrated by way of example and not limitation and the figures of the accompanying drawings in which like references denote like or corresponding parts, and in which:

FIG. 1 is a front view of the adult toy device in accordance with the preferred embodiment of the present invention.

FIG. 2 is a front view of the adult toy device of FIG. 1 being positioned in the female anatomy.

FIG. 3 is a front view of the adult toy device of FIG. 1 with dimensions.

FIG. 4 is a side view of the adult toy device of FIG. 1.

### DETAILED DESCRIPTION OF THE PREFERRED EMBODIMENT AND THE DRAWINGS

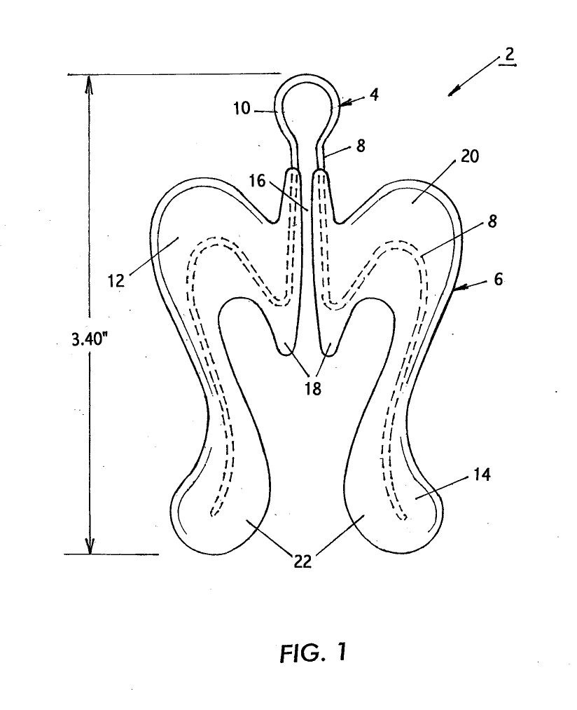

As shown in FIG. 1, an adult toy device 2 designed for the female anatomy is provided by the present invention. In accordance with the preferred embodiment, device 2 includes a clip-on portion 4 and a labia expanding portion 6. The entire device 2 is preferably made of a flexible material. Further, the labia expanding portion 6 has a resiliency with respect to the clip-on portion 4. Both portions are preferably formed as parts of the same unitary device.

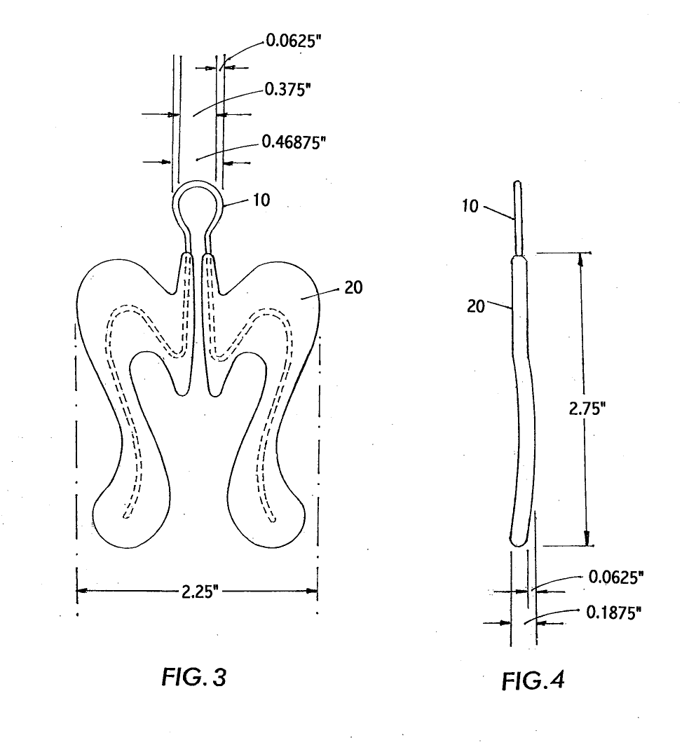

The clip-on portion 4 preferably includes an elongated ring 10 and ribs 8 extending from the ring 10 into the body of the labia expanding portion 6. The ring is located at the top of device 2 and is intended to clip onto the clitoris by surrounding the clitoris from the top, sliding the device down and along both sides of the clitoris, thus extruding the clitoris. As shown in FIGS. 1-3, in the preferred embodiment, ring 10 has a slightly elongated oval shape and is preferably 0.65 inches high and 0.47 inches wide, has a width of opening of approximately 0.37 inches and wall thickness of approximately 0.06 inches. Alternative configurations of the ring 10 are possible. For example, the ring may be formed as having a substantially circular opening.

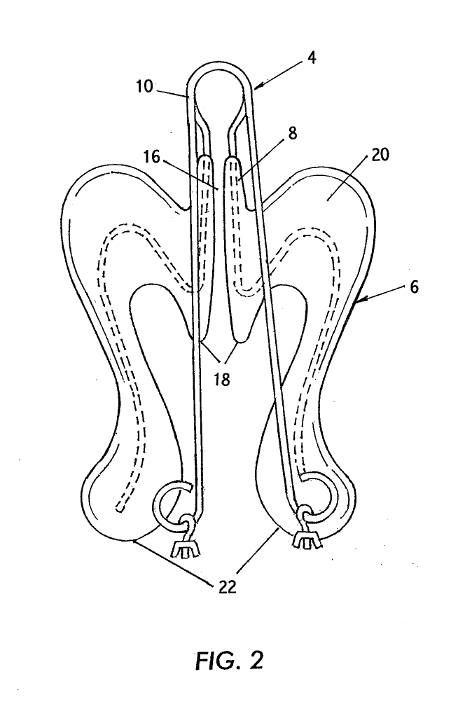

As shown in FIGS. 1 and 2, in its final positioning, ring 10 is located around the clitoris such that its walls are positioned adjacent to the top and the sides of the clitoris. Further, because the aperture of the ring 10 is formed as an elongated circular opening, i.e., as shown in FIG. 2, the walls of the ring are also partially located under the clitoris. Finally, because ribs 8 attached to the ring 10 have some resiliency, the ultimate position of the ring results in the walls of the ring being slightly behind the clitoris, thus, isolating and extruding the clitoris within the aperture of the ring.

As shown in FIGS. 1-4, the labia expanding portion 6 of the device preferably includes a wing section 12 and a finger-gripping section 14. The wing section is located immediately below and adjacent to the clip-on portion 4. In accordance with the preferred embodiment, wing section 12 includes a pair of flexible wings 20, an expansion gap 16 and a pair of lips 18. Lips 18 are formed at the internal edges of the wings 20 such that lips 18 are placed into contact with labia walls when the device is installed. Resilient ribs 8 of the clip-on portion extend into wings 20 substantially following the contour of the lips 18 and the remainder of the wings. The expansion gap 16 is located between the two lips 18. Ribs 8 are preferably constructed such that they tend to expand the expansion gap 16 by pushing the wings 20 away from each other. Depending on a particular woman's anatomy, wing section 12 may be 2.75 inches high, 2.25 inches wide and approximately 0.18 inches thick. When the device 2 is properly installed, lips 18 and wings 20 are in contact with labia's folds, pushing them back to separate the labia and to expose the opening of the vagina within the expansion gap 16. The ultimate position of the device will force the labia to remain separated.

As shown in FIGS. 1-2, the finger gripping section 14 includes a pair of finger grips 22. In order to install the device, a user would squeeze the finger grips 22 towards each other; position the clip-on portion 4 behind and around the clitoris, extruding and isolating it; then, position lips 18 inside the vaginal opening next to the labia walls; and release the finger grips. Because resilient ribs 8 are constructed to push the wings 20 away from each other, when finger grips 22 are released, lips 18 push against the labia walls expanding the gap 16 and exposing the opening of the vagina within the gap.

In the preferred embodiment, the device is made of a combination of silicone, rubber, and plastic composite materials rendering the device flexible but capable of providing stability once fastened.

Although the present invention has been described in relation to particular embodiments thereof, many other variations and modifications and other uses will become apparent to those skilled in the art. It is preferred, therefore, that the present invention be limited not by the specific disclosure herein, but only by the appended claims.

1. An adult toy device, comprising:
a clip-on portion having a ring positionable so as to surround and extrude a female clitoris;
a wing portion attached to the clip-on portion, the wing portion having a pair of lips positionable adjacent to labia walls, a gap formed between the lips, and a pair of wings extending from the lips and having a resiliency; and
a pair of finger gripping regions, each finger gripping region being positioned at one of the wings.

2. The adult toy device according to claim 1, wherein the clip-on portion further comprises a substantially oval aperture surrounded by the ring, the female clitoris being extruded within the oval aperture.

3. The adult toy device according to claim 1, wherein the clip-on portion further comprises a substantially circular aperture surrounded by the ring, the female clitoris being extruded within the substantially circular aperture.

4. The adult toy device according to claim 1, further comprising a resilient rib positioned within the wing portion following a contour of the lips and the wings, the resilient rib being attached to the ring of the clip-on portion and being configured so as to expand the gap between the lips by pushing the wings apart from each other.

5. A method of enhancing female orgasm, comprising:
providing an adult toy device including a clip-on portion having a ring, a wing portion having a pair of lips, a gap formed between the lips, a pair of wings extending from the lips, and a pair of finger gripping regions;
squeezing the wings of the adult toy device by pinching the finger gripping regions toward each other;
positioning the ring of the clip-on portion around female clitoris so as to surround and extrude it;
positioning the lips of the wing portion adjacent to labia walls; and
releasing the finger gripping regions so as to push the lips against the labia walls, expanding the gap between the lips and exposing a vaginal opening within the gap.

6. A device for enhancing female clitoral stimulation, comprising:
a clip-on portion having a ring positionable around a female clitoris and a substantially oval aperture surrounded by the ring, the ring and the aperture being configured so as to extrude the female clitoris within the oval aperture;
a wing portion flexibly attached to the clip-on portion, the wing portion having a pair of lips positionable adjacent to labia walls, a gap formed between the lips, and a pair of wings extending from the lips and having a resiliency;
a pair of finger gripping regions, each finger gripping region being positioned at one of the wings, the wings being movable with respect to each other and the clip-on portion by applying force to the finger gripping regions; and
a resilient rib positioned within the wing portion following a contour of the lips and the wings, the resilient rib being attached to the ring of the clip-on portion and being configured so as to expand the gap between the lips by pushing the wings apart from each other.
